(19) United States
(12) Patent Application Publication
(10) Pub. No.: US 2017/0044223 A1
(43) Pub. Date: Feb. 16, 2017

(54) Human Sperm Fibrous Sheath (FS) Proteins: New Target Antigens For Use In Therapeutic Cancer Vaccines And Diagnostic Screening And Methods Of Using Same

(71) Applicant: Kiromic, LLC, Lubbock, TX (US)

(72) Inventors: Maurizio Chiriva-Internati, Lubbock, TX (US); Everardo Cobos, Lubbock, TX (US); Wijbe Martin Kast, La Canada Flintridge, CA (US); Kenneth Morrow, JR., Newport, KY (US)

(73) Assignee: Kiromic, LLC, Lubbock, TX (US)

(21) Appl. No.: 15/094,726

(22) Filed: Apr. 8, 2016

Related U.S. Application Data
(63) Continuation of application No. 12/421,356, filed on Apr. 9, 2009, now abandoned.

(51) Int. Cl. C07K 14/47

(52) U.S. Cl. CPC C07K 14/47; A61K 39/00

(57) ABSTRACT

Cancer vaccines were demonstrated to be promising strategies for cancer treatment; however these strategies are limited by the paucity of target antigens that provoke an effective immune response. The Inventors of the present patent application propose that sperm fibrous sheet proteins constitute a new class of potential antigens for use in the development of effective, durable cancer vaccines. This hypothesis is supported by the expression of the sperm fibrous sheath protein, known as Sperm protein 17 (Sp17), which is detected in tumors of unrelated histological origin. It has the ability to induce T-cell based immune responses. The expression of the Sperm protein 17 (Sp17) in tumors of unrelated histological origin, and its' natural localization in the human sperm fibrous sheath (FS), led the Inventors to hypothesize that FS proteins might represent a new potential class of target antigens useful for developing cancer vaccines, as well as diagnostic and therapeutic treatments.

### FIELD OF THE INVENTION

This disclosure relates to tumor associated antigens and genes and antibodies. More specifically, this disclosure relates to human sperm fibrous sheath proteins and (genes, antibodies) for therapeutic and diagnostic propose and methods of using the same.

### BACKGROUND OF THE INVENTION

Cancers, or malignant tumors, which are characterized by continuous cell proliferation and cell death, can be classified into three categories: carcinomas, sarcomas, and leukemia. Conventional treatment approaches for malignant tumors are highly invasive and sometimes have only a palliative effect. Increased efforts have been made to apply immunomodulatory strategies, such as those employing tumor associated antigens (TAAs), in antitumr or treatment.

TAAs confer an immunological distinctness to tumor cells that differentiate them from the majority of normal cells and provide diagnostic and therapeutic targets for human cancers. Several monoclonal antibodies have been identified which react specifically with cancerous cells such as T-cell acute lymphoblastic leukemia and neuroblastoma.

TAAs are predominantly expressed in normal gametogenic tissues as well as in different histological types of tumors. In testis, TAAs are expressed exclusively in cells of the germ cell lineage, although there is a marked variation in the protein expression pattern during different stages of sperm development. Likewise, a heterogeneous expression is observed in tumors. The methylation status of the promoter region seems to be the main, but not the only,

Cancer vaccines have been demonstrated to be a promising strategy for cancer treatment but the strategy is limited by the paucity of target antigens that provoke an effective immune response. We propose that sperm fibrous sheet proteins constitute a new class of potential antigens for cancer vaccines. This hypothesis is supported by the expression of the sperm fibrous sheath protein known as Sperm protein 17 detected in tumors of unrelated histological origin. It has the ability to induce T-cell based immune responses.

The expression of the Sperm protein 17 (Sp17) in tumors of unrelated histological origin, and its natural localization in the human sperm fibrous sheath (FS), has led us to hypothesize that FS proteins might represent a new potential class of target antigens useful for developing cancer vaccines.

The flagellum of the mammalian spermatozoa consists of four distinct segments: a) the connecting piece adjacent to the head; b) the middle piece defined by a tightly packed helical array of mitochondria surrounding the cytoskeletal structures of the flagellum; c) the principal piece, and d) the short end piece.

The major cytoskeletal structures are the axoneme, also present in cilia, and the outer dense fibers and FS, which are unique to spermatozoa. The FS is a unique cytoskeletal entity, which underlies the plasma membrane, surrounds the axoneme and outer dense fibers, and defines the extent of the principal piece of the sperm flagellum [1]. It consists of two longitudinal columns connected by closely arranged semicircular ribs that assemble from distal to proximal throughout spermatogenesis [1,2]. Although the function of FS is unclear, it is believed to serve as a scaffold for both glycolytic enzymes and constituents of the signaling cascades, and it is well positioned to play a role in the regulation of sperm motility [1].

Several proteins localized in the FS have been identified, including Sp17, CABYR, AKAP-3, AKAP-4, TAKAP-80, Rhopilin, Ropporin, GSTM5. There are no doubts numerous others. Of these Sp17 and CABYR have been thoroughly analyzed:

Sperm protein 17: wider expressed than originally thought A family of tumor-associated antigens, called cancer-testis (CT) antigens has been found in a limited number of normal human tissues and various human tumors of unrelated histological origin [2]. One of these, Sp17, has been identified as a CT antigen in multiple myeloma, other blood malignancies, and ovarian cancer. An mRNA encoding Sp17 was detected in 17% of patients with multiple myeloma and in the primary tumor cells from 70% of patients with primary ovarian carcinoma [3,4]. At the protein level, Sp17 has been found in human germinal cells of the testis (except in the case of spermatogonia) [5], the ciliated epithelia of the respiratory airways, and both the male and female reproductive systems [6]. It has also recently been found in the synoviocytes of females affected by rheumatoid arthritis [7] and the melanophages of cutaneous melanocytic lesions [8], as well as in a proportion of primary nervous system tumors [9] and a subset of esthesioneuroblastomas [10]. As it is expressed in germinal cells and various neoplastic tissues, Sp17 is more widely distributed in humans than originally thought. Although the function of Sp17 is still unknown, the high degree of sequence conservation throughout its N-terminal half, and the presence of an A-kinase anchoring protein (AKAP)-binding motif within this region, suggests that it might play a regulatory role in a protein kinase A (PKA)-independent AKAP complex in both germinal and neoplastic cells.

Pharmaceutically useful composition comprising the Fibrous Sheath (and or anything that is part of all it) gene antibodies and or proteins from it, product may be formulated according to known methods such as by admixture of a pharmaceutically acceptable carrier. This carrier suitably contains minor amounts of additives such as substances that enhance the isotonicity and chemical stability. Such materials are non toxic.

### SUMMARY OF THE INVENTION

Disclosed herein is a Fibrous Sheath. Further disclosed herein is a therapeutic regimen comprising introduction of a gene encoding at least a portion of an AKAP for the treatment or prevention of a disorder or disease state. Further disclosed herein is a method for the treatment of multiple myeloma, ovarian cancer, breast, lung, CML, ALL, Leukemia, B and T cell disease and Immunological disease, pancreas cervical colon, prostate, brain cancer and other disease. Further disclosed herein is a method for the treatment of solid tumors. Further disclosed herein is a method for the treatment of non-solid tumors. Further disclosed herein is a method for the diagnosis of multiple myleoma comprising obtaining human sera and contacting the antibody with the sera and detecting antibody binding to an antigen wherein the antigen comprises at least a portion of a polypeptide having parts all compounds of the Fibrous sheath. Further disclosed herein is a vaccine comprising a gene encoding at least a portion of a Fibrous sheath protein. Since the first cloning of a human tumor antigen [11], the identification and development of immunogenic cancer vaccines targeting these antigens has represented a formidable task [12, 13, 14]. Further disclosed herein is a diagnostic and therapeutic regimen comprising introduction of a gene encoding at least a portion of a Fibrous sheath for the treatment or prevention of a disorder or disease state.

Introduction of a Fibrous Sheath to an organism may induce an immune response that functions to protect the organism from a disorder or disease state (e.g., malignant tumor) or may be a component of a therapeutic regimen for the treatment of a disorder or disease state. Hereinafter, compositions comprising a gene encoding a Fibrous Sheath polypeptide or fragment thereof or a Fibrous Sheath polypeptide or fragment thereof for use as an immunogen is termed an immunogenic Fibrous Sheath composition (IFSC).

In an embodiment, a IIFSC comprises fragments or portions of the disclosed nucleic acid molecules are also encompassed by the present disclosure. By “fragment” or “portion” is meant less than full length of the nucleotide sequence. As used herein, an “isolated” nucleic acid molecule is a nucleic acid molecule that is separated from other nucleic acid molecules that are usually associated with the isolated nucleic acid molecule. Thus, an isolated nucleic acid molecule includes, without limitation, a nucleic acid molecule that is free of sequences that naturally flank one or both ends of the nucleic acid in the genome of the organism from which the Isolated nucleic acid is derived (e.g., a cDNA or genomic DNA fragment produced by per or restriction endonuclease digestion). Such an isolated nucleic acid molecule is generally introduced into a vector (e.g., a cloning vector, or an expression vector) for convenience of manipulation or to generate a fusion nucleic acid molecule as will be described in more detail later herein. In addition, an isolated nucleic acid molecule can include an engineered nucleic acid molecule such as a recombinant or a synthetic nucleic acid molecule.

As indicated, nucleic acid molecules of the present disclosure which encode a Fibrous Sheath may include, but are not limited to those encoding the amino acid sequence of the mature polypeptide, by itself; and the coding sequence for the mature polypeptide and Additional sequences such as a pre-, or pro- or pre pro-protein sequence. Also encoded by nucleic acids of the disclosure are the Fibrous Sheath sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those, which provide additional functionalities. Thus, the sequence encoding the polypeptide may be fused to a marker sequence, such as a sequence encoding a peptide which facilitates purification of the fused polypeptide.

Further embodiments of the present disclosure include isolated nucleic acid molecules that comprise a polynucleotide having a nucleotide sequence at least 90% identical, and alternatively at least 95%, 96%, 97%, 98% or 99% identical, to a nucleotide sequence encoding a polypeptide of the Fibrous sheath nucleotides sequence from blast, or a polynucleotide which hybridizes under stringent hybridization conditions to a polynucleotide encoding the polypeptide sequence of Fibrous Sheath. This polynucleotide, which hybridizes, does not hybridize under stringent hybridization conditions to a polynucleotide having a nucleotide sequence consisting of only A residues or of only T residues.

In an embodiment, the IFSC comprises variants of the nucleic acid molecules of the present disclosure, which encode portions, analogs or derivatives of the Fibrous Sheath protein. Variants may occur naturally, such as a natural allelic variant. By an “allelic variant” is intended one of several alternate fonts of a gene occupying a given locus on a chromosome of an organism. Non-naturally occurring variants may be produced using art-known mutagens is techniques. Other such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. In an embodiment, Fibrous Sheath variants suitable for use in this disclosure comprise conservative or silent substitutions, additions and deletions, which do not alter the properties and activities of Fibrous Sheath or portions thereof.

In an embodiment, the IFSC comprises an isolated nucleic acid molecule comprising a polynucleotide which encodes the amino acid sequence of an epitope-bearing portion of an Fibrous Sheath having the polypeptide sequence of the Fibrous Sheath.

In an embodiment, the IFSC comprises a nucleic acid molecule encoding a Fibrous Sheath polypeptide or fragment thereof as a component of a recombinant vectors. Additional vector components and methods of preparing such vectors are known to one of ordinary skill in the art.

In an embodiment, the IFSC comprises at least a portion of a Fibrous sheath polypeptide. Polypeptides of the present disclosure include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. The polypeptide or a fragment thereof may be produced by any means known to one of ordinary skill in the art for the production of such molecules. For example, Fibrous Sheath can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. For example, a method of producing a protein may comprise growing a cell comprising a nucleic acid vector comprising a nucleic acid molecule, comprising a sequence of nucleotides that encodes the polypeptide of Fibrous Sheath under conditions whereby the protein is expressed and isolating the protein.

In another aspect, the IFSC comprises a peptide or polypeptide comprising an epitope-bearing portion of a Fibrous Sheath. The epitope of this polypeptide portion is an immunogenic or antigenic epitope of. An “immunogenic epitope” is defined as a part of a protein that elicits an antibody response when the whole protein is the immunogen. On the other hand, a region of a protein molecule to which an antibody can bind is defined as an “antigenic epitope.” The epitope-bearing peptides and polypeptides of the present disclosure may be produced by any means known to one of ordinary skill in the art.

We hypothesize that the sperm FS protein, Sp17 constitutes a new class of target antigens for developing cancer vaccines. This model is currently under investigation [15]. The present hypothesis will increase the number of available target antigens in cancer vaccines, and add new understanding to the relationship between human germ-cell development and carcinogenesis [2, 16, 17].

In an embodiment, epitope-bearing peptides and polypeptides of the typed disclosed herein may be used to induce antibodies according to methods well known in the art. Immunogenic epitope-bearing peptides of the type disclosed herein, those parts of a protein that elicit an antibody response when the whole protein is the immunogen, are identified according to methods known in the art.

In an alternative embodiment, the IFSC may comprise antibodies that are specific to a Fibrous Sheath polypeptide or a fragment thereof. It is not intended that the present disclosure be limited to a particular type of antibody as such both polyclonal and monoclonal antibodies are contemplated. Such antibodies may be made in a variety of animals [e.g., rabbits, horses, cows (e.g., in the milk), and birds. Also contemplated are human and “humanized” antibodies.

Fibrous Sheath-specific antibodies for use in the present disclosure can be raised against the intact Fibrous Sheath protein or an antigenic polypeptide fragment thereof. As used herein, the term “antibody” (Ab) or “monoclonal antibody” (Mab) is meant to include intact molecules as well as antibody fragments (such as, for example, Fab and F(ab′)2 fragments), which are capable of specifically binding to Fibrous Sheath. Fibrous Sheath-antibodies (of part of it) may be prepared by any of a variety of methods. For example, cells expressing the Fibrous Sheath protein or an antigenic fragment thereof can be administered to an animal in order to induce the production of sera containing polyclonal antibodies. Alternatively, a preparation of Fibrous Sheath is prepared and purified to render it substantially free of natural contaminants. Such a preparation is then introduced into an animal in order to produce polyclonal antisera of greater specific activity. Alternatively, the antibodies of the present disclosure are monoclonal antibodies (or Fibrous Sheath binding fragments thereof). Such monoclonal antibodies can be prepared using hybridoma technology which are techniques known to one of ordinary skill in the art.

A method for using AKAP to induce immune responses in vivo, in a vertebrate such as a mammal, including a human, may comprise: isolating at least one Fibrous Sheath gene, linking the gene to regulatory sequences such that the gene is operatively linked to control sequences which, when introduced into a living tissue direct the transcription initiation and subsequent translation of the gene, and introducing the gene into a living tissue. The introduction of the Fibrous Sheath polypeptide or a fragment thereof into an organism may induce an immune response. In an embodiment, the organism may be experiencing a disorder or diseased state, such as for example a proliferative disorder and the immune response may function to ameliorate the disorder or diseased state. Alternatively, the immune response may function prophylatically (e.g., as a vaccine) to prevent the onset of a disorder or diseased state. In an embodiment, the method may be used for the treatment or prevention of a proliferative disorder comprising a solid or non-solid tumor. Alternatively, the method may be used for the treatment or prevention of multiple myeloma and ovarian cancer and leukemia, breast, lung cancer.

In an embodiment, a method of detecting multiple myeloma in an organism may comprise obtaining sera from the organism and contacting the sera with an antibody raised against one or more Fibrous Sheath epitopes. Binding of the antibody may be used as an indicator of the presence of the Fibrous Sheath polypeptide and may serve as a diagnostic tool for the detection of multiple myeloma. Methods of binding and detection of antibody binding are known to one of ordinary skill in the art.

Pharmaceutically useful compositions comprising the Fibrous Sheath gene or gene product may be formulated according to known methods such as by the admixture of a pharmaceutically acceptable carrier. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents EDTA; sugar alcohols such as mannitol or sorbitol; counterions such as sodium; and/or nonionic surfactants such as polysorbates, poloxamers, or PEG.

Therapeutic or diagnostic compositions of the present disclosure (e.g., IFSC) may be administered to an individual in amounts sufficient to treat or diagnose a disease or disorder. The effective amount may vary according to a variety of factors such as the individual's condition, weight, sex and age. Other factors include the mode of administration. The pharmaceutical compositions may be provided to the individual by a variety of routes such as subcutaneous, topical, oral and intramuscular.

In an embodiment, the IFSC is a component of a vaccine for the treatment or prevention of a proliferative disorder such as multiple myeloma ovarian, lung, prostate, pancreas, breast, leukemia and other disease. The vaccines of the present disclosure may comprise DNA that encode recombinant Fibrous Sheath that contain the antigenic determinants that induce the formation of neutralizing antibodies in the human host. Such vaccines are also safe enough to be administered without danger of clinical infection; do not have toxic side effects; can be administered by an effective route; are stable; and are compatible with vaccine carriers. The vaccines may be administered by a variety of routes, such as orally, parenterally, subcutaneously or intramuscularly. The dosage administered may vary with the any number of factors such as sex, weight, and age of the individual; and the route of administration. The vaccine may be used in dosage forms such as capsules, suspensions, elixirs, or liquid solutions. The vaccine may be formulated with an immunologically acceptable carrier. The vaccines are administered in prophylactically or therapeutically effective amounts, that is, in amounts sufficient to generate an immunologically protective response. The effective amount may vary according to the type of disorder and/or diseased state. The vaccine may be administered in single or multiple doses.

The disclosure also provides a pharmaceutical pack or kit comprising one or more containers filled with one or more of the ingredients of the pharmaceutical compositions of type described herein. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present disclosure may be employed in conjunction with other therapeutic compounds.

While embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.1 0 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference herein is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

### EXAMPLES

The invention having been generally described, the following examples are given as particular embodiments of the invention and to demonstrate the practice and advantages thereof. It is understood that the examples are given by way of illustration and are not intended to limit the specification or the claims in any manner.

1. The future application is having new targets for tumor vaccines and cancer diagnostics through the development of antibodies against antigens localized in the fibro-sheath.

2. This will be the first anatomical structure used as a protein to induce a better vaccine pulsing in dendritic cells or using just the single Fibrous sheath as a structure that can stimulate protection against this class of antigens that are expressed in different tumors such as ovarian, lung, prostate, breast, colon, pancreas cancers and multiple myeloma, as well as other hematological and non hematological tumors. It will be used to active the immune system in vivo and ex vivo application.

3. Novelty is the anatomical structure to be used for the first time as a protein, genes, and antibodies to stimulate tumor immunity.

4. We can develop novel reagents such as different antigens, genes protein from this structure and antibodies by micro-array applications.

5. The sperm fibrous sheath proteins that constitute a potential group of cancer testis antigens.

6. The use of the fibrous sheath (genes and proteins and antibodies derived from the fibrous sheath), an anatomical structure, used as a protein to stimulate tumor immunity.

7. A cancer-related antigen, proteins genes and antibodies derived from the fibrous sheath, for example the Sperm Protein 17, AKAP-4, ASP, CABIR, PTTG-1, ROPPORIN and all others potential proteins, genes and antibodies derived from a fibrous sheath component which is a cancer testis antigen occurring in a number of malignant cells and some normal cells.

8. A cancer-related antigen, proteins genes and antibodies derived from the fibrous sheath, Sperm Protein 17, AKAP-4, ASP, CABIR, PTTG-1, and ROPPORIN, which will be used to develop a therapeutic cancer vaccine.

9. A cancer-related antigen, proteins genes and antibodies derived from the fibrous sheath, for example Sperm Protein 17, AKAP-4, ASP, CABIR, PTTG-1, ROPPORIN, which will be used to develop a diagnostic test for cancer.

10. A cancer-related antigen, proteins genes and antibodies derived from the fibrous sheath, for example antibodies directed against the Sperm Protein 17, AKAP-4, ASP, CABIR, PTTG-1, ROPPORIN and all other potential genes, proteins, antibodies for use in therapeutic and diagnostic test applications.

11. An immunogen comprising an isolated FIBROUS SHEATH protein or fragment thereof as described herein.

12. A therapeutic regimen comprising introduction of a gene encoding at least a portion of a FIBROUS SHEATH for the treatment or prevention of a disorder or disease state as described herein.

13. A method for the treatment of multiple myeloma, ovarian, cervical, lung, prostate breast, colon leukemia, and other disease of the type described herein.

14. A method for the treatment of solid tumors of the type described herein.

15. A method for the treatment of non-solid tumors of the type described herein.

16. A method for the diagnosis of multiple myeloma, cervical , breast, prostate, leukemia ovarian, lung, pancreas, colon comprising obtaining human sera and contacting the antibody with the sera and detecting antibody binding to an antigen wherein the antigen comprises at least a portion of a polypeptide having a nucleotides sequence of the Fibrous Sheath.

17. A vaccine comprising a gene encoding at least a portion of a Fibrous Sheath protein.
